(19) United States
(12) Patent Application Publication
(10) Pub. No.: US 2009/0069433 A1
(43) Pub. Date: Mar. 12, 2009

(54) NASAL RINSE ADDITIVE

(76) Inventor: Wayne Jeffrey Perry, Schenectady, NY (US)

Correspondence Address: AMIN HALLIHAN, LLC, 444 NORTH ORLEANS STREET, SUITE 400, CHICAGO, IL, 60654, US

(21) Appl. No.: 12/207,753

(22) Filed: Sep. 10, 2008

Related U.S. Application Data
(60) Provisional application No. 60/971,216, filed on Sep. 10, 2007.

(51) Int. Cl. A61K 31/135

(52) U.S. Cl. 514/627

(57) ABSTRACT

A nasal rinse additive composition includes oleoresin capsicum containing capsaicin, dihydrocapsaicin, nordihydrocapsaicin, homodihydrocapsaicin, and homocapsaicin as active ingredients. The nasal rinse additive composition can further include vegetable glycerin, purified water, spearmint oil, lemongrass oil, sea salt, and/or ascorbic acid. The composition can be used to relieve and/or prevent pain, dryness, and/or inflammation associated with chronic and occasional nasal congestion.

This application claims the benefit of earlier filed U.S. Patent Application Ser. No. 60/971,216 filed on 10 Sep. 2007.

### FIELD OF THE INVENTION

The present invention relates to a nasal rinse additive composition containing capsaicinoids for relieving nasal congestion, headache, inflammation, and general sinus pain along with providing antibacterial properties. The present invention is related to a copending application, U.S. Ser. No. 11/731,656, entitled SINUS RELIEF COMPOSITION AND METHOD OF PRODUCING THE SAME, filed by the inventor with the United States Patent Office and which disclosure is incorporated herein.

### BACKGROUND OF THE INVENTION

Capsaicinoids are the active components in chili peppers and plants belonging to the Capsicum family. The pungency level of a plant belonging to the Capsicum family is measured on the Scoville scale. The greater the number of Scoville heat units indicates the “hotter” the plant. Capsaicin is a capsaicinoid and the most prevalent capsaicinoid in chili peppers, followed by dihydrocapsaicin, nordihydrocapsaicin, homodihydrocapsaicin, and homocapsaicin. Pure capsaicin ranges between 15,000,000-16,000,000 Scoville units. Dihydrocapsaicin is an irritant and has a pungency similar to the range of capsaicin. Nordihydrocapsaicin, homodihydrocapsaicin, and homocapsaicin are also irritants and have a pungency range of about 8,600,000-9,100,000 Scoville units.

Each capsaicinoid and its corresponding chemical structure is shown below.

| | |
|---|---|
| Capsaicin | 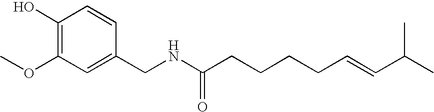 |
| Dihydrocapsaicin | 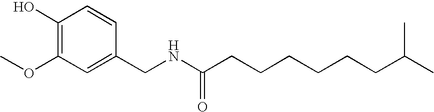 |
| Nordihydrocapsaicin | 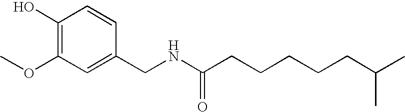 |
| Homodihydrocapsaicin | 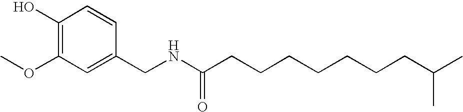 |
| Homocapsaicin | 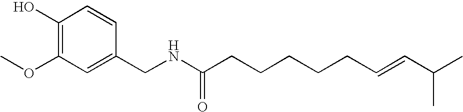 |

Capsaicinoids are irritants and produce a sensation of burning or hotness when they come in contact with human tissue. Capsaicinoids chemically interact with sensory neurons by binding to the thermoreceptor nerve endings inside the body or to receptors in the skin. These receptors can be stimulated with heat or physical abrasion thereby causing chemical signals to pass through the cell membrane and into the cell which causes the neuron to generate its own signal to the brain. The capsaicinoid molecules induce the same effect of producing a burning sensation, but not a chemical burn, that heat or physical abrasion does by binding to the thermoreceptor nerve endings. Therefore, capsaicinoids stimulate the nerve endings which leads to desensitization, a process that strategically provides an analgesic effect that suppresses pain and inflammation more quickly and for a longer period of time than other analgesics.

Capsaicinoids have many uses such as a heat additive for foods, topical ointments, and non-lethal weapons. Capsaicinoids are used in foods to add spice or “heat”, such as in hot sauce or salsa. Capsaicinoids are also used as a topical ointment to relieve minor aches and pains in muscles and joints, such as symptoms associated with arthritis. Capsaicinoids are also used as an ingredient in the non-lethal weapon commonly known as “pepper spray”, which when sprayed into the eyes or onto the skin is painful to the recipient.

Current treatments for relief of nasal congestion include a variety of saline and/or water based nasal rinses, and/or prescription and over-the-counter drug allergy medicines. However, these current treatments may be ineffective for severe congestion, may not relieve related symptoms and may require a prescription for utilization.

### SUMMARY OF THE INVENTION

One object of the present invention is for a natural, effective composition for the relief of general nasal congestion.

Another object of the present invention is a composition having components which provide a synergistic effect of destroying germs and bacteria that cause nasal congestion and related symptoms.

Still another object of the present invention is for a composition which provides relief of nasal congestion symptoms along with headache, inflammation and related general sinus pain.

A further object of the present invention is a composition for the relief of severe, chronic and occasional nasal congestion symptoms having the beneficial effects of capsaicinoids without the lingering pain and discomfort of pure pepper.

In accordance with the present invention, a nasal rinse additive composition containing capsaicinoids is used for fully relieving and preventing nasal congestion symptoms, including pain, headache, and inflammation has been developed. In one form of the present invention, the nasal rinse additive composition has been developed to be added to a nasal rinse and administered intranasally.

The nasal rinse additive composition in accordance with the present invention includes oleoresin capsicum including capsaicin, dihydrocapsaicin, nordihydrocapsaicin, homodihydrocapsaicin, and homocapsaicin as active ingredients that has been developed specifically for the purpose of relieving chronic and occasional nasal congestion symptoms. Additionally, the nasal rinse additive composition relieves symptoms associated with nasal congestion, such as headache, inflammation and general sinus pain.

In one embodiment of the present invention, the nasal rinse additive composition includes about 0.009% to about 1.0% oleoresin capsicum per the total water weight of the base.

In another embodiment of the present invention, the nasal rinse additive composition includes oleoresin capsicum including capsaicin, dihydrocapsaicin, nordihydrocapsaicin, homodihydrocapsaicin, and homocapsaicin as active ingredients and having a heat range of 100,000 to 500,000 Scoville units.

In yet another embodiment of the present invention, the nasal rinse additive composition includes oleoresin capsicum including about 67-71% capsaicin, about 20-24% dihydrocapsaicin, about 5-9% nordihydrocapsaicin, about 0.25-2% homodihydrocapsaicin, and about 0.25-2% homocapsaicin.

In still another embodiment of the present invention, the oleoresin capsicum may be water soluble.

Additionally, the nasal rinse additive composition can also include spearmint oil, lemongrass oil, vegetable glycerin, sea salt, purified water, and ascorbic acid.

In another embodiment of the present invention, about 1 mL to about 10 mL of the nasal rinse additive composition is added per 4 ounces of water based nasal rinse and administered intranasally.

These and other embodiments of the present invention are more fully described in connection with the detailed description.

### DETAILED DESCRIPTION OF THE INVENTION

The present invention relates to a nasal rinse additive composition containing capsaicinoids used for fully relieving and preventing chronic and occasional nasal congestion symptoms, including pain, inflammation, and infection. The present composition is designed to be added to a nasal rinse and administered intransally.

One embodiment of the present invention relates to a nasal rinse additive composition having oleoresin capsicum including capsaicin, dihydrocapsaicin, nordihydrocapsaicin, homodihydrocapsaicin, and homocapsaicin as active ingredients, combined with spearmint oil, lemongrass oil, vegetable glycerin, sea salt, purified water, and ascorbic acid.

The synergistic effect of the components of the nasal rinse additive composition of one form of the invention relieves chronic and occasional nasal congestion symptoms without the lingering pain and discomfort of pure pepper. The nasal rinse additive composition of one embodiment of the present invention also relieves related symptoms, such as those associated with colds, flu, throat infections, bacterial infections, post nasal drip and strained throat muscles.

Oleoresin capsicum, a natural resin, is a concentrated extract of capsaicinoids from several Capsicum varieties. The Capsicum variety utilized in the present invention is derived from cayenne pepper plants, which includes Capsicum baccatum and Capsicum frutescens. The oleoresin capsicum includes capsaicin, dihydrocapsaicin, nordihydrocapsaicin, homodihydrocapsaicin, and homocapsaicin as active ingredients and the main heat component of Capsicum variety peppers. Preferably, the oleoresin capsicum utilized in the present invention is all natural, water soluble or soluble in a liquid suspension material. It is within the scope of the present invention that a combination of synthetic capsaicinoids including about 67-71% capsaicin, about 20-24% dihydrocapsaicin, about 5-9% nordihydrocapsaicin, about 0.25-2% homodihydrocapsaicin, and about 0.25-2% homocapsaicin could also be used. Additionally, powdered capsicum, capsicum tincture, capsicum oil, capsicum pepper juice and/or mixtures thereof could be utilized in the present invention.

The capsaicinoids in the oleoresin capsicum deplete the chemicals which cause pain and swelling, such as Substance P which is responsible for transmitting pain in the nervous system. The depletion of these chemicals relieves nasal congestion, the associated inflammation and general sinus pain.

The oleoresin capsicum relieves nasal congestion symptoms by flushing out, for example, bacteria, excess mucous, and allergens including dust mites, mold, fungi and pollen. The oleoresin capsicum stimulates the nerve endings in the lining of the nasal passages, sinus passages, and trigeminal nerve network thereby causing the nerve endings to swell to facilitate release of the toxins, bacteria, excess mucous, and allergens, which relieves the nasal congestion symptoms. The oleoresin capsicum provides also antiseptic properties and kills bacteria which are associated with throat infections. The oleoresin capsicum further provides relief for severe and chronic nasal congestion which is not provided by saline and/or water based nasal rinses alone.

The spearmint oil cleanses the nasal passages, provides a clean flavor and aroma, and acts as a carrier for the oleoresin capsicum.

The vegetable glycerin is a natural preservative which protects and stabilizes the nasal rinse additive composition. The vegetable glycerin also assists the capsaicinoids contained in the oleoresin capsicum to maintain their potency and effectiveness for extended periods of time. The vegetable glycerin maintains maximum effectiveness of the nasal rinse additive composition by drawing moisture to the lining of the nasal passage which assists in relieving dryness. Additionally, the vegetable glycerin shortens the length of time of any burning sensation associated with the capsaicinoids in the oleoresin capsicum without reducing its effectiveness. The vegetable glycerin stabilizes the formulation, lengthens the shelf life, does not promote the formation of yeast in the composition, shortens the length of time of the burning sensation associated with the capsaicinoids and preserves the potency of the capsaicinoids for up to five times longer than other natural preservatives, such as aloe vera.

The ascorbic acid naturally preserves the nasal rinse additive composition by adjusting the pH levels. Additionally, the ascorbic acid (vitamin C) supports the immune system and helps to prevent and flush out bacteria growth that causes nasal congestion and related symptoms.

The sea salt naturally preserves and stabilizes the nasal rinse additive composition and provides minerals for cleansing the nasal passage.

The lemongrass oil cleanses the nasal passages, provides a clean flavor and aroma, and acts as a carrier for the oleoresin capsicum

Finally, the purified water dilutes the nasal rinse additive composition and permits the nasal rinse additive composition to secrete directly into the human body's natural water in the nasal passages.

In another embodiment of the present invention, the nasal rinse additive composition may be added to a nasal rinse and administered or dispensed intranasally. The nasal rinse additive is added to a nasal rinse mixture, such as saline/water and plain water. The nasal rinse additive is added to a nasal rinse by means such as a dropper, squeeze applicator, or spoon. The nasal rinse may be administered intranasally, for example, by neti pot, nasal douching application, nasal lavage application, nasal rinse bottle, nasal syringe, battery or electric nasal application, and/or bulb syringes.

In a further embodiment of the present invention, about 1 mL to about 10 mL of the nasal rinse additive composition is added per 4 ounces of water based nasal rinse and administered intranasally.

In accordance with the present invention, the nasal rinse additive composition includes about 1.75 mL oleoresin capsicum per 5 gallons of purified water base or liquid suspension material. The nasal rinse additive composition may include about 0.009% to about 1.0% oleoresin capsicum per the total water weight of the base. The nasal rinse additive composition may include oleoresin capsicum including about 67-71% capsaicin, about 20-24% dihydrocapsaicin, about 5-9% nordihydrocapsaicin, about 0.25-2% homodihydrocapsaicin, and about 0.25-2% homocapsaicin as active ingredients. Also, the nasal rinse additive composition may include oleoresin capsicum including capsaicin, dihydrocapsaicin, nordihydrocapsaicin, homodihydrocapsaicin, and homocapsaicin as active ingredients and possessing a heat range of to 500,000 Scoville units. The oleoresin capsicum including capsaicin, dihydrocapsaicin, nordihydrocapsaicin, homodihydrocapsaicin, and homocapsaicin is present in a non-caustic and safe amount.

In other embodiments of the present invention, the oleoresin capsicum may be water soluble, the spearmint may be an essential oil, the lemongrass may be an essential oil, the sea salt may be solarized, the vegetable glycerin may be derived from coconuts and palms, the ascorbic acid may be synthetic, and the purified water may be steam distilled to closely match the body's natural water makeup.

The following example of one embodiment of the present invention relates to a nasal rinse additive composition having oleoresin capsicum in 5 gallons of purified water or liquid suspension material, and demonstrates the scope of the present invention. The nasal rinse additive composition comprises:
about 1.75 mL oleoresin capsicum;
about 2 mL spearmint oil;
about 2.25 mL lemongrass oil;
about 700 mL vegetable glycerin;
about 100 mL sea salt; and
about 100 mL ascorbic acid.

Another example of the present invention relates to a nasal rinse additive composition in 5 gallons of purified water or liquid suspension material, including:
about 0.009% to about 1.0% by weight of the total water weight of oleoresin capsicum;
about 0.01% to about 0.02% by weight of the total water weight of spearmint oil;
about 0.01% to about 0.02% by weight of the total water weight of lemongrass oil;
about 0.5% to about 0.75% by weight of the total water weight of sea salt;
about 3.0% to about 3.75% by weight of the total water weight of vegetable glycerin; and
about 0.5% to about 0.75% by weight of the total water weight ascorbic acid.

Another embodiment of the present invention is a nasal rinse additive kit having a nasal rinse additive composition having oleoresin capsicum including capsaicin, dihydrocapsaicin, nordihydrocapsaicin, homodihydrocapsaicin, and homocapsaicin as active ingredients, combined with vegetable glycerin, sea salt, purified water, spearmint oil, lemongrass oil, and ascorbic acid; and an instruction for a human to administer the nasal rinse additive composition intranasally through a nasal rinse. One form of the nasal rinse additive kit includes the nasal rinse additive composition described above.

While in the foregoing specification the invention has been described in relation to certain preferred embodiments thereof, and many details have been set forth for the purpose of illustration, it will be apparent to those skilled in the art that the invention is susceptible to additional embodiments and that certain details described herein can be varied considerably without departing from the basic principles of the invention.

We claim:

1. A nasal rinse additive composition, comprising:
a liquid suspension material, and
oleoresin capsicum including capsaicin, dihydrocapsaicin, nordihydrocapsaicin, homodihydrocapsaicin, and homocapsaicin as active ingredients.
